(19) United States
(12) Patent Application Publication
(10) Pub. No.: US 2010/0100002 A1
(43) Pub. Date: Apr. 22, 2010

(54) APPARATUS FOR DIAGNOSIS AND MONITORING OF HEPATIC STEATOSIS BASED ON ELECTRICAL IMPEDANCE MEASUREMENT

(76) Inventors: Rosa Villa Sanz, Barcelona (ES); Antoni Guimera Brunet, Barcelona (ES); Damia Parramon Capdevila, Barcelona (ES); Ivan Erill Sagales, Barcelona (ES); Antoni Ivorra Cano, Barcelona (ES); Jordi Aguilo Llobet, Barcelona (ES)

Correspondence Address: DARBY & DARBY P.C., P.O. BOX 770, Church Street Station, New York, NY, 10008-0770, US

(21) Appl. No.: 12/443,934

(22) PCT Filed: Nov. 30, 2007
(86) PCT No.: PCT/IB07/03753

§ 371 Date: May 13, 2009

(30) Foreign Application Priority Data
Oct. 3, 2006 (ES) P200602518

(51) Int. Cl. A61B 5/05

(52) U.S. Cl. 600/547

(57) ABSTRACT

The present invention relates to a system for the rapid determination of the degree of hepatic steatosis (fat in the liver) via a direct measurement of the hepatic electrical impedance at one or more frequencies. The measurement is taken by means of surface or minimally invasive sensors, which can be coupled to other devices for medical use (e.g., laparoscopic probes). By means of an interpolation algorithm based on correlations between impedance and the percentage of hepatic fat in reference biopsies, the system is capable of determining the degree of hepatic steatosis in the organ measured immediately and without requiring any other kind of intervention. This permits its application to procedures such as liver transplant, allowing a rapid diagnosis of viability to be made, along with other surgical procedures and on explanted organs.

### SECTOR OF THE ART

The present invention relates to the medical/health sector and more specifically to medical recording and diagnostic devices. In particular, this invention relates to the use of devices based on the measurement of electrical impedance in order to determine the degree of hepatic steatosis rapidly and non-invasively, its main proposed application being for the diagnosis of steatosis of livers intended for organ transplant.

### STATE OF THE ART

### Definition of Hepatic Steatosis

Hepatic steatosis (HS) is the accumulation of histologically visible lipids in the cytoplasm of hepatocytes (liver cells), and is the most frequent metabolic alteration at the hepatic level. Among other causes, HS can be due to an increase in the intake (greater ingestion) of fats, a decrease in β-oxidation, a decrease in proteins or the ingestion of a toxin. In Western society, the most frequent underlying causes are obesity and alcohol.

### Determination of Hepatic Steatosis

The histological examination by means of hepatic puncture-biopsy (HPB) is the most accepted medical technique for the diagnosis of HS and therefore constitutes the reference standard for the diagnosis of this disease. Even so, HPB presents certain drawbacks. On the one hand, the technique is invasive and its field of examination is small, a fact that complicates the diagnosis of HS given that it can display very heterogeneous patterns in its occurrence. On the other hand, the processing and analysis time is long and requires expert pathologists, resulting in a process that is expensive and partially subjective.

Depending on the percentage of liver cells (hepatocytes) affected, HS is classified as: 1) slight: fewer than 25% of hepatocytes affected; 2) moderate: from 25% to 50% of hepatocytes affected; and 3) severe: more than 50% of hepatocytes affected. Moreover, in HS two main morphological categories can be distinguished: macrovesicular HS and microvesicular HS, with both being able to be found in a single case of HS.

### Hepatic Steatosis and Liver Transplant

Historically, HS is associated with a highly significant post-transplant mortality and morbidity. A HS higher than 15% (above all in the case of being macrovesicular), being over the age of 65 or being a carrier of the hepatitis C virus are three fundamental and independent parameters which most strongly mark the expectation of survival of the hepatic organ or of the transplanted patient.

Nowadays, due to the progressive increase in the need for liver donors, the number of donors being accepted who are elderly and, as a consequence, with a larger number of associated pathologies, such as obesity or alcoholism, is increasing. This fact is leading to an increase in the number of livers with a greater or lesser degree of steatosis that are being used for liver transplant.

The use of livers with steatosis is associated with an increase in hepatic Primary Non-Function (PNF) following transplant. This non-function is related to the fact that steatosic livers are less resistant than normal livers to lesions induced by the processes of ischaemia/reperfusion and cold storage that are inherent to the transplant process.

In this respect, HS with major and clear disturbance is one of the main causes of rejection of the graft for transplant. The diagnosis comes from the data of the donor's clinical history, analysis of the level of enzymes, visual inspection, ultrasound, and, on a few occasions, from HPB. The main problem of HS in transplants lies in detecting degrees of steatosis that are not so evident, since it is sometimes during the surgical procedure (laparoscopy) when the decision is taken on whether or not to use the organ as a graft in view of a suspicious appearance of HS (yellowish surface).

HPB is clearly the most reliable test for the diagnosis of HS among those currently done, but its drawbacks are accentuated when it is carried out during the surgical process of transplanting. The need to carry out several invasive punctures in order to avoid the local effect of HS and the long processing time are factors which go against the basic needs of the transplant process: a rapid and minimally invasive diagnosis. Moreover, in the specific case of the transplant, HPB can generate uncertain results owing to the techniques of rapid cooling used in preservation, which complicates the interpretation by the pathologist.

Because of all this, the rapid and reliable quantification of hepatic grafts currently continues to be an unsolved problem and one of great concern in liver transplant procedures.

### Electrical Impedance in the Characterisation of Live Tissues

The term “electrical impedance” defines the relation between the alternating voltage applied to a sample and the resulting electrical current. In the particular case in which the sample forms part of a living being, the term “electrical bioimpedance” or simply “bioimpedance” is normally used. Electrical bioimpedance does not in itself constitute a physiological parameter having a direct interpretation. Nevertheless, the bioimpedance values, or the passive electrical properties mathematically deriving from them, can indirectly reflect certain states and events that are of interest in medical practice.

The values obtained from the measurement of the electrical impedance depend on the geometry of the sample and on the electrical properties of the material composing the sample. In a sample composed of various parts, the geometrical relations among those parts will to a large degree determine the values obtained. In fact, there exists an important group of medical techniques which base their functioning on the influence which geometrical changes at the macroscopic scale, produced in various anatomical compartments, entail for the bioimpedance. Some examples belonging to this group are impedance cardiography, impedance plethysmography and impedance pneumography. Another technique, known as “Bioimpedance Analysis” (BIA), bases its functioning on the fact that fatty tissue displays a much higher electrical resistivity than lean tissue. The measure of impedance permits the percentage of water in the body to be determined and, thanks to this fact and to its correlation with various additional parameters, such as the weight and sex of the subject, the proportion of body fat can be derived in relation to lean tissue and other tissues (such as bone) present in the body by means of mathematical formulas and reference values.

In this regard, the present invention differs from those cited above in that its aim is not to reveal any geometric characteristic at the macroscopic scale. Here, it is assumed that one is dealing with a single macroscopically homogenous tissue and that the parameters derived from the bioimpedance will primarily depend on the electrical characteristics proper to the tissue submitted for analysis. In other words, the bioimpedance will be used for characterising the state of live tissue. Various physical and chemical properties of live tissues cause them to present a certain impedance which not only distinguishes among them but also permits certain pathological states to be detected and evaluated as deviations from normality. There exist earlier inventions proposing the use of impedance for detecting the presence and evaluating the state of certain pathologies in live tissue, such as cancer (US2006100488, US2005065418, EP1600104, WO03084383, US2003105411), ischaemia (WO2004105862, U.S. Pat. No. 5,807,272, WO0114866) and even caries (U.S. Pat. No. 6,230,050). Nevertheless, to date no proposal has been made for the use of electrical impedance for evaluating the degree of steatosis in organs nor has any scientific study being conducted in this regard.

### Physiological Model and Measurement of Electrical Impedance

It is generally considered that the cell membrane of living beings acts as a fine layer of imperfect dielectric material which separates two electrolytic media (extracellular medium and intracellular medium). This model to a large degree explains the common characteristics of living tissues in terms of impedance in relation to other materials. Any factor modifying any of the parameters of this model will have an impact on some of the values obtained by means of electrical impedance. Numerous cellular or histological alterations have been described that modify the impedance values, such as: cellular oedema, extracellular oedema, necrosis, closure or opening of intercellular ionic bonds, physical decoupling of cells, ionic imbalance, changes in the shape of cells and opening of pores in the membrane by means of electroporation.

For each frequency analysed, electrical impedance provides a pair of values. This pair can be expressed as the “magnitude” and “phase” of the impedance. Nevertheless, other pairs of values mathematically related to the magnitude and phase and are commonly used (for example, “real part” and “imaginary part” of the impedance, or “conductivity” and “permittivity”). All these values, individually or mathematically combined, can be used for characterising the tissue. Mathematical models based on experience (for example, the Cole model) are frequently used for minimising the number of values required in the characterisation of live tissues.

There exist a range of techniques for obtaining the passive electrical properties of live tissues. For high frequencies (>100 MHz) methods based on the transmission or reflection of electromagnetic waves are normally used. For lower frequencies methods can be used based on inductive coupling though much more common are measurement methods based on electrodes. In many cases a measurement by means of four electrodes is chosen (tetrapolar or Kelvin method) though measurements using two or three electrodes are nevertheless also possible. The measurement techniques of bioimpedance and their applications are found described in depth in the book “Bioimpedance and Bioelectricity Basis” by S. Grimnes and O. G. Martinsen, published in the year 2000 by Academic Press (London, UK).

### DESCRIPTION OF THE INVENTION

The present invention is based on the fact, observed by the inventors, that the measurement of electrical impedance can be used for detecting and evaluating the degree of steatosis in the liver of a living being. This fact is totally novel since, as mentioned above, there exist inventions that propose the use of impedance for detecting and evaluating certain pathologies in living tissues, but none of them makes reference to steatosis.

Based on this discovery, the objective of the present invention is to provide measuring equipment or apparatus for biomedical applications, both in clinical practice and in experiments with animals, which can be used for the diagnosis of hepatic disturbances due to the accumulation of fat, known as hepatic steatosis (HS).

Therefore, an object of the present invention is an apparatus for clinical use for the diagnosis of steatosis in the liver, hereinafter inventive apparatus for detecting steatosis, characterised in that it measures the electrical impedance of the hepatic organ in order to determine its degree of steatosis. Clinical use apparatus is understood to be any apparatus designed for medical and veterinary applications and which is therefore sterilisable, biocompatible and electrically safe.

A particular objective of the invention is the inventive apparatus for detecting steatosis which comprises at least:
a) an impedance measurement sensor formed from various biocompatible electrodes,
b) a measurement module of the electrical impedance between 10 Hz and 10 MHz and
c) an electronic device for control and analysis of the measurements obtained.

This apparatus determines the percentage of fat in a liver (steatosis) by means of measuring the impedance, carried out with electrodes in contact with that organ and relating it to the degree used in clinical practice.

In organs affected with hepatic steatosis, and the way in which, as a consequence of this pathology, the fat accumulates inside the cell, changes take place progressively in the structure of the tissue (primarily, an increase in cell size and a reduction in the extracellular space) which alter the electric impedance of the tissue at different frequencies, permitting a discrimination in the degree of hepatic steatosis based on the direct measurement of impedance. In measurements at one or more frequencies (multifrequential) of the impedance, differences are clearly observed between normal and fatty hepatic tissue, with these differences increasing linearly as a function of the percentage of fat accumulated, and allowing the possibility of applying a reliable correlation with the degree of HS determined histologically.

As has already been mentioned, the measurement of the electrical impedance can be carried out at one or more frequencies. Although measurement at a single frequency (typically a low frequency) is theoretically valid for the detection of steatosis in the liver, such measurement is subject to a high variability owing to the notable physiological variation among patients. In this sense, obtaining a multifrequential measurement notably improves the results with regard to diagnosis, since it permits a multitude of indirect parameters to be extracted which display a lower variability and, consequently, greater diagnostic reliability. These parameters can be simple relations, such as for example the relation among moduli or the difference between high and low frequencies, or more complex parameters resulting from the fit to the model of the impedance spectrum (alpha, central frequency, R0 and R∞).

In this regard, another particular object of the invention is the inventive apparatus for detecting steatosis characterised by the fact that it comprises means for measuring the hepatic electrical impedance at various frequencies swept in the range lying between 10 Hz and 10 MHz and uses relations among the values measured at different frequencies to estimate the degree of hepatic steatosis.

The measurement of electrical impedance implies the injection of an electrical current into the medium and measuring the simultaneous reading of the resulting electrical potential. In this regard, the number, material, form and arrangement of electrodes are extremely important in measuring the electrical impedance since they respectively affect the measurement technique thereof, its efficiency and the volume of tissue measured. Likewise, and particularly for clinical applications, the form and arrangement of the electrodes will also determine the invasive nature of the impedance measurement, and the material used for those electrodes will define their biocompatibility.

In this regard, the measurement sensor for the impedance which the inventive apparatus for detecting steatosis contains can have an arrangement of:
a) Surface sensor consisting of electrodes which can be of different sizes and forms,
b) Minimally penetrating or minimally invasive sensor. In this case, the electrodes are placed in small needles which penetrate the liver producing a minimal lesion. The system can have from one needle with four electrodes to having four needles with a single electrode each

So, a particular embodiment of the invention is the inventive apparatus for detecting steatosis which comprises electrodes as non-invasive surface sensors, and another particular embodiment of the invention is the inventive apparatus for detecting steatosis which comprises electrodes placed in small needles as minimally invasive penetrating sensors.

As far as the material is concerned, another particular embodiment of the invention is the inventive apparatus for detecting steatosis which comprises electrodes made of and/or coated with gold, silver, silver chloride, platinum or any other biocompatible material with high conductance as impedance measurement sensors.

With regard to the measurement technique and arrangement of the electrodes, another particular embodiment of the invention is the inventive apparatus for detecting steatosis which comprises a sensor consisting of surface and/or penetrating electrodes in which the number of electrodes lies between 2 and 4. These configurations of electrodes permit measurements of the impedance to be made at two, three and four points depending on the method used. Configurations are also permitted such as measurements at three points with the earth of the electric scalpels acting as the earth terminal.

Likewise, another particular embodiment of the invention is the inventive apparatus for detecting steatosis which comprises multiple (more than 4) surface and/or penetrating electrodes on a semi-rigid or flexible substrate which can be independently selected in twos, threes or fours for measuring the impedance at various spatial points on the surface of an organ.

The inventive apparatus for detecting steatosis is also adaptable to the diagnosis of the degree of hepatic steatosis, rapidly and non-invasively, in hepatic organs that are going to be transplanted, in patients subjected to laparotomy, in other abdominal pathologies not associated with organ transplant, as well as in other surgical operations not requiring a laparotomy but in which the surface of the liver can be accessed, such as via probes or catheters by means of procedures of minimally invasive abdominal surgery.

So, another particular object of the invention is the inventive apparatus for detecting steatosis in which the electrodes are incorporated in a single device for facilitating their handling manually during the laparotomy (open abdominal surgery). FIG. 1 represents an apparatus for detecting steatosis designed for this application.

Another particular object of the invention is the inventive apparatus for detecting steatosis in which the electrodes are incorporated into medical laparoscopy devices for being located in probes or catheters which these devices incorporate and which are introduced into the open cavity during laparoscopy operations (FIG. 2).

Another particular object of the invention is the inventive apparatus for detecting steatosis in which the electrodes are located on a platform which weighs the organ and which is used once the liver has been extracted. This adaptation of the inventive apparatus for detecting steatosis is designed for the extraction of the hepatic organ during organ transplant procedures and in procedures of forensic medicine. In this embodiment, of course, there do not exist any connections by means of external wires, instead the connection between the electrodes and the circuitry of the process is made on the same platform. FIG. 3 shows this design of inventive apparatus for detecting steatosis.

The inventive apparatus for detecting steatosis can incorporate a temperature sensor and/or a pressure sensor for adjusting the impedance values obtained and thereby avoid the measurement of the electrical impedance from being distorted by variations in the pressure applied on the electrodes or in the temperature of the tissue being measures or of the ambient temperature (FIG. 5).

In this regard, another particular object of the invention is the inventive apparatus for detecting steatosis characterised in that it comprises temperature sensors and a measurement module for temperature in order to adjust the impedance measurements and prevent the values obtained from being affected by temperature variations in the tissue to measure or in the ambient temperature.

Likewise, another particular object of the invention is the inventive apparatus for detecting steatosis characterised in that it comprises a pressure sensor and a measurement module for pressure in order to adjust the impedance measurements and prevent the values obtained from being affected by the pressure between the sensor device and the organ.

As has been mentioned above, the present invention provides a diagnostic procedure characterised by the following stages:
Coupling the impedance sensor of the inventive device for detecting steatosis to the hepatic tissue for measurement of the electrical impedance,
Measuring the impedance, at one frequency at least, within the range 10 Hz-10 MHz,
In the event that the system has temperature sensors, correcting the values of the impedance as a function of the temperature,
In the event that the system has pressure sensors, automatically correcting the excess pressure or informing on whether the pressure exerted on the surface is correct when carrying out the impedance measurement,
Calculating the parameters that identify the state of the organ on the basis of the impedance measurements, and
Estimating the degree of steatosis with the measurements of electrical impedance made with respect to known average parameters of organs with and without different degrees of steatosis extract from a population sample.

The measurements with the inventive apparatus can be easily repeated at different points of a hepatic organ, which is an important fact given the heterogeneous nature of the occurrence of fat in the hepatic tissue.

The degree of steatosis presented by the liver is obtained by means of weighting the parameters of the different variables by means of a mathematical expression. The base parameters are obviously the values of modulus, phase, real part and imaginary part of the impedance at different frequencies, but the system makes extensive use of the relations among the base parameters in order to generate more robust parameters that are less dependent on the variability among organs, resulting from the fit to the model of the impedance spectrum.

To the parameters obtained, a correlation algorithm is applied which also permits other parameters of the patient affecting the impedance to be incorporated, such as age, weight and sex, along with other pathologies of that particular patient, and also parameters inherent to the vital state of the organ, is such as its degree of blood perfusion, its preservation with liquids at low temperature or in situations of explantation, etc. The algorithm can make a linear or non-linear correlation, depending of the degree of precision and number of parameters to correlate, and the correlation variables can be adjusted manually by any expert or using any automatic regression method.

The advantages of this procedure for the diagnosis of steatosis with regard to hepatic biopsy (HPB, the reference model) consist of the fact that the inventive apparatus for detecting steatosis:
Is not invasive (it does not require tissue extraction).
Does not leave any lesion nor subsequent fibrotic sequelae.
Offers an immediate diagnosis.
Permits various measurements to be carried out easily and quickly in order to cover the entire area of the liver and not just taking a point sample, thus avoiding the effect of localisation and the consequent error due to heterogeneity of the hepatic fatty occurrence.
The data recorded is automatically correlated with the degrees of habitual clinical use of steatosis, facilitating its interpretation.

In this regard, another object of the invention makes reference to the use of apparatus that measure the electrical impedance in order to detect and determine the degree of steatosis in the hepatic organ in diagnostic procedures.

Within the range of applications, another particular object of the invention consists of the use of apparatus or devices that measure the electrical impedance as a procedure for detection of steatosis in human liver.

Another particular object of the invention is the use of apparatus that measures the electrical impedance as a procedure for detection of steatosis in animal liver.

Another particular object of the invention is the use of devices that measure the electrical impedance as a procedure for detection of steatosis in surgical processes for organ transplant.

Another particular object of the invention is the use of devices that measure the electrical impedance as a procedure for detection of steatosis in processes of laparotomy (open abdominal surgery) and laparoscopy (minimally invasive abdominal surgery).

Another particular object of the invention is the use of devices that measure the electrical impedance as a procedure for detection of steatosis in procedures of forensic medicine.

Another particular object of the invention is the use of devices that measure the electrical impedance as a procedure for detection of steatosis in food control procedures in animals.

### DESCRIPTION OF THE FIGURES

FIG. 1 shows the inventive apparatus for detecting steatosis. The sensors (2) are placed in contact with the liver (1) and are connected by means of a wire to the electronic measurement system (3). The data can be sent to and stored in external devices such as a computer (4) or an electronic agenda (PDA) (5). This arrangement is appropriate for use in open abdominal surgery (laparotomy).

FIG. 2 shows a diagram of the arrangement of sensors of the inventive apparatus for detecting steatosis for use on laparoscopy, in which the sensors have been included in the probe (1) of a medical device for laparoscopy and connected to the electronic unit of the apparatus by means of a wire (2) and the corresponding connector (3).

FIG. 3 shows a diagram of an inventive apparatus for detecting steatosis on a weight measurement system for clinical use on livers explanted in forensic medicine or other applications with explanted livers. The apparatus incorporates the electrodes for measurement of impedance (1) and a 10K3A1B temperature sensor manufactured by BETATHERM (2).

FIG. 4 shows an electronic diagram of the inventive apparatus for detecting steatosis corresponding to the example described. The apparatus takes a measurement of the impedance at 4 points and consists of the following elements: the contacts of the electrodes (I+, V+, V−, I−), where a current is injected which circulates between the electrodes I+ and I−, and the voltage is picked up between the electrodes V+, V−, (1) a current generator which injects current between the external electrodes (I+ and I−), (2) a current to voltage converter which measures the injected current, (3) an instrumentation amplifier which measures the voltage between the internal electrodes, (4) an analogue-digital signal converter which converts the signals to discrete values and finally (5) a control unit which performs the calculation of the impedance and calculates the degree of steatosis.

FIG. 5 shows an arrangement of sensors. Included in this case are: (1) four electrodes as impedance sensor, (2) a 10K3A1B temperature sensor manufactured by BETATHERM and (3) an FSG pressure sensor manufactured by Honeywell. These sensors are connected by means of a wire (4) to the electronic unit, and make up the inventive apparatus for detecting steatosis.

FIG. 6 shows an arrangement of sensors for performing a more extensive measurement of the impedance in the hepatic organ and, consequently, for carrying out a quick overall evaluation of the degree and distribution of the hepatic steatosis. The sensor (2) consists of a flexible array of electrodes covering a large zone of the liver (1). The measurement equipment (3) interrogates different configurations of electrodes and performs an analysis of the impedance measures in an overall manner.

FIG. 7 shows a Nyquist representation of the multifrequential impedance measurement in livers, where the solid line is the result obtained with a non-steatosic liver and the broken line is the result from a steatosic liver. The axes of the graph represent the real part (ordinate axis) and the imaginary part (abscissa axis) of the impedance. The lowest frequencies appear on the right of the graph and the highest on the left.

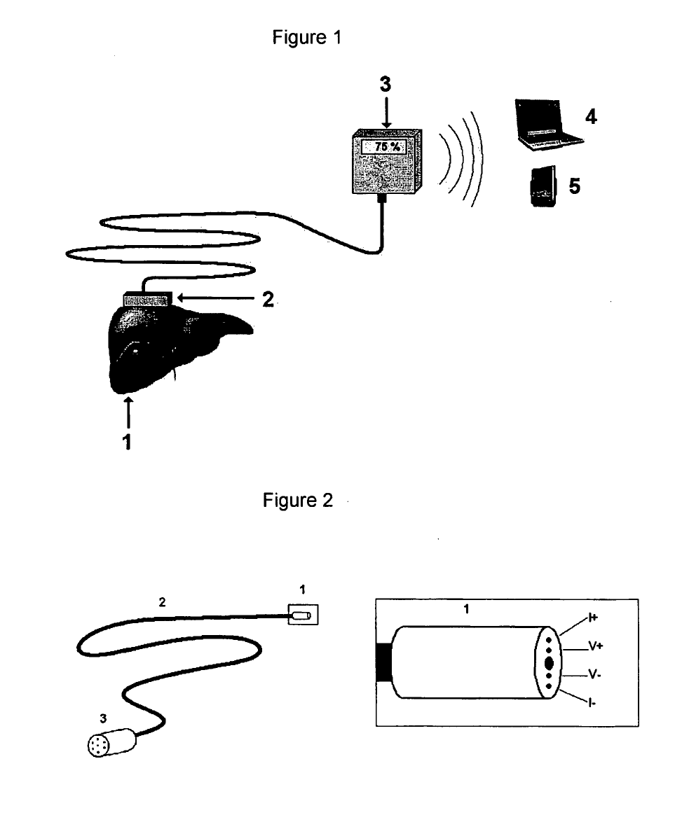

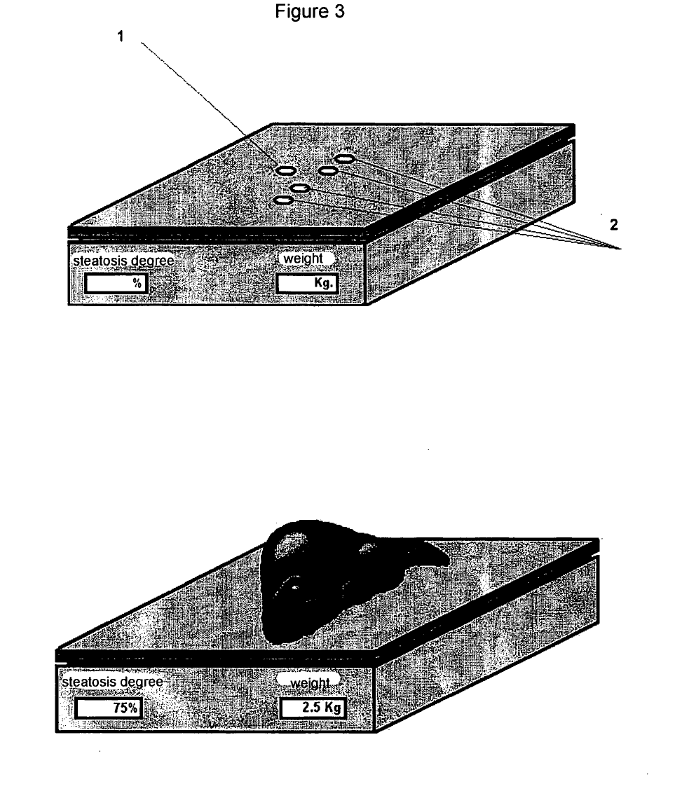

### EXAMPLE OF EMBODIMENT OF THE INVENTION

The following example of embodiment describes the inventive apparatus for measuring steatosis and the methodology of use in the context of its application to the liver transplant. The objective of the application in the scope of liver transplant of the inventive apparatus for measuring steatosis is to permit a rapid diagnosis of the degree of hepatic steatosis with the aim of aiding the surgeon in the decision to discard or accept a liver donated for the transplant.

### Description of the Inventive Apparatus for Measuring Steatosis

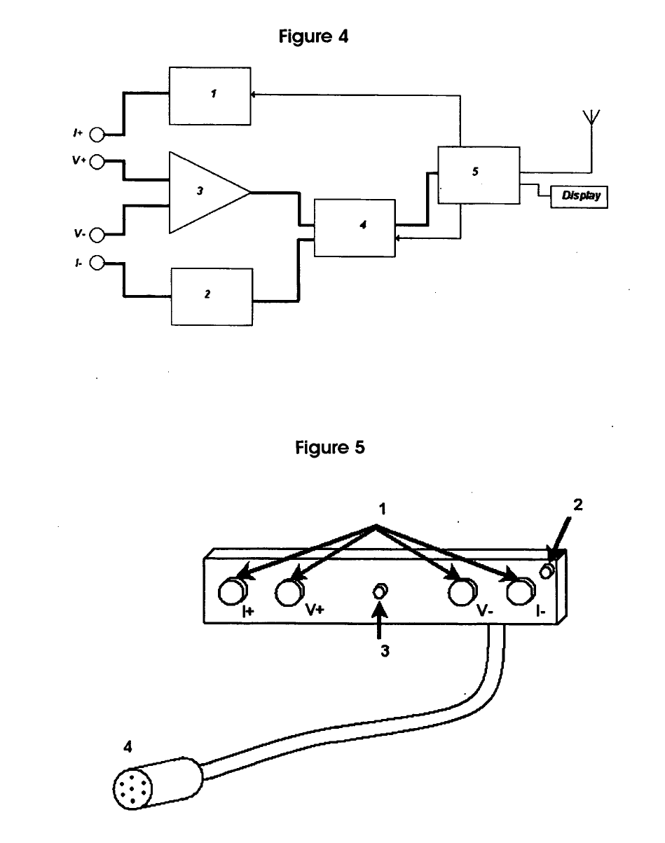

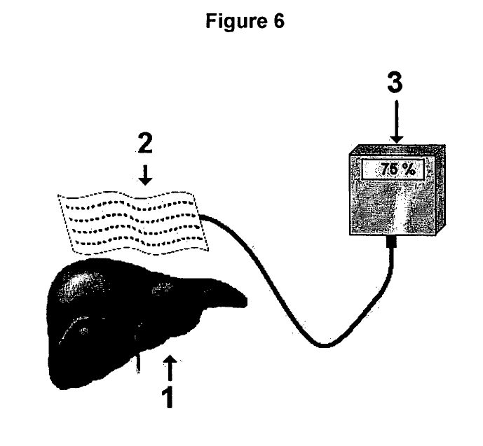

In this example, the inventive apparatus for measuring steatosis produced by the inventors is used with the following arrangement (FIG. 4):
An impedance measurement sensor, formed from 4 gold electrodes (E-TEC S.A., reference: SIB-120-S037-22) of diameter 0.64 mm and with separations of: 2.54 mm between 1+ and V+; 7.62 mm between V+ and V−, and 2.54 mm between V− and I−. The height of the electrodes is 2 mm, with the rest of the electrode being coated with a Epoxy OG147-7 from EPO-TEK. These electrodes are connected to the measurement device by means of wires.
A measurement device consisting of: (1) a module formed from a DDS AD9835 generator which generates a sinusoidal signal of between 1 and 100 kHz which is transmitted via the electrode I+. Another module (2) which is responsible for picking up the current entering I− using an AD844 amplifier which generates a voltage proportional to the current measured. Another module (3) which picks up the voltage between the two internal electrodes (V+ and V−) using an AD8130 instrumentation amplifier. Another module (4), formed from an AD9243 analogue-digital converter responsible for digitalising the two voltages read.
An MB90F583B micro-controller from Fujitsu (4), which controls the analogue-digital conversion model, is responsible for calculating the impedance value on the basis of the digitised values of voltage and current, it performs the final conversion to degree of steatosis by means of an interpolation algorithm based on tables (see below) stored in the memory of the micro-controller and manages the control of buttons and the different modes of operation.
An LCD VI303-DPRC display from Varitronix (5), and various buttons for manipulating the device.

### Description of the Methodology of Use

### Initialisation

The procedure for using the device is very simple. Once the abdominal opening has been made and the liver can be seen, the inventive apparatus for measuring steatosis is activated by pressing the activation button. Instants after being activated, the inventive apparatus for measuring steatosis shows on the display that it is ready for use. A choice can then be made between two modes of functioning: single measurement and multiple measurements, by pressing the mode button, and between two modes of recording; monofrequential and multifrequential, by pressing the frequency button.

### Manipulation and Measurements

With the device activated and the functioning mode selected, the electrodes of the inventive apparatus for measuring steatosis are placed in contact with the hepatic surface. With the electrodes in their place, the button to start reading is pressed and, instants afterwards, the display shows a percentage reflecting the degree of hepatic steatosis. If the multiple readings mode has been chosen, these readings are done sequentially, pressing the mode button after each reading, relocating the electrodes in another part of the liver and again pressing the button to start reading. At the end of the final reading, the mode button is pressed twice and the apparatus shows the degree of steatosis determined on the basis of the average of the readings made in the different locations.

### Calculation of the Degree of Hepatic Steatosis and Diagnosis

Shown below is the method of calculation of the degree of hepatic steatosis using the multi- and monofrequential modes for this specific embodiment of the inventive apparatus for measuring steatosis.

### Multifrequential Mode

In the multifrequential mode the inventive apparatus for measuring steatosis calculates the degree of hepatic steatosis by means of an algorithm that uses the multifrequential impedance values expressed according to a Nyquist representation. This representation of the multifrequential impedance permits the algorithm to obtain certain maximum and minimum values of the real part of the impedance in the range of frequencies sampled. The algorithm then performs the division between the values of the real part at minimum and maximum frequency, and it obtains what is known as the factor Z—real, which is then correlated with the degrees of hepatic steatosis.

$$factor Z - real = \frac{{Z}_{F min}}{{Z}_{F max}}$$

This correlation, included in the algorithm as an interpolation table, has been obtained experimentally by means of many different assays conducted on livers of Sprague-Dawley rats, with a control group and a study group. The rats in the study group were subjected to a feeding protocol with fat-rich and hyperprotein diets, provoking steatosis in them. Following measurement of the impedance, a histological study was then made of the biopsies of the livers used. The histological classification of the fatty livers was carried out on the basis of the number of globules of fat per field, and the samples examined were classified according to the following categories: without steatosis, slight steatosis <10%, moderate steatosis 10%-25%, and severe steatosis 25%-60%. This classification was then correlated with the measurement of impedance, which was taken in the form of factor Z—real, generating the following interpolation table:

TABLE 1
Experimental table of correlation between the factor derived from
measurement of impedance and the hepatic steatosis percentage.

| Factor: | Hepatic Steatosis Percentage |
|---|---|
| <3 | <10% (normal) |
| 3-5 | 10%-25% (slight steatosis) |
| 5-7 | 25%-60% (moderate steatosis) |

### Monofrequential Mode

In the case of taking a measurement at a single frequency, the device only makes one measurement of impedance at a frequency of 1 kHz, and the impedance value obtained is correlated in a specific table for measurements at a single frequency, obtained during the same experimentation process as described above for the multifrequency case.

TABLE 2
Experimental table of correlation between the measurement of
impedance at a single frequency and the hepatic steatosis percentage.

| Factor: | Hepatic Steatosis Percentage |
|---|---|
| <1000 | <10% (normal) |
| 1001-2500 | 10%-25% (slight steatosis) |
| 2501-3200 | 25%-60% (moderate steatosis) |

### Example of Use

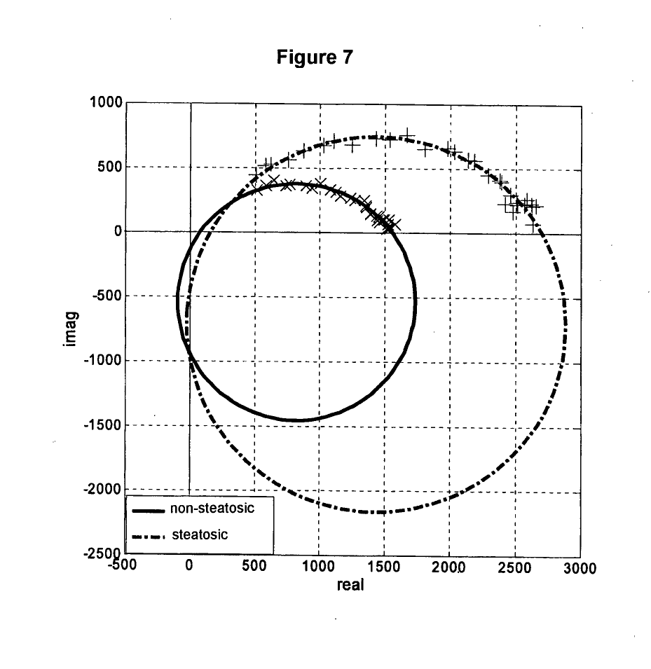

By way of illustration, FIG. 7 presents two experimental results obtained with this implementation of the inventive apparatus for measuring steatosis in the mode of a single multifrequency reading on rat livers. Also shown is its use for the diagnosis of hepatic steatosis and the subsequent assistance in taking the decision to reject/accept the organ in transplants.

### Case 1

The first case, represented by the solid line in FIG. 7, is the result of impedance measurements in a rat liver from the control group (i.e., non-steatosic). In this case the inventive apparatus for measuring steatosis calculated a value of 3 for the factor Z—real.

$$factor Z - real = \frac{{Z}_{F min}}{{Z}_{F max}} = \frac{1500 Ω}{500 Ω} = 3$$

Using the calculated factor Z—real, the inventive apparatus for measuring steatosis correlated this value with the interpolation table shown above (Table 1) and generated an indicative value of the degree of steatosis: <10 (NORMAL).

### Case 2

Following with the example of FIG. 7, the second case (represented by the data in a broken line of FIG. 7) corresponds to the results obtained in a rat liver from the study group (i.e., a steatosic liver). For the factor Z—real, the inventive apparatus for measuring steatosis calculated a value of 5.2:

$$factor Z - real = \frac{{Z}_{F min}}{{Z}_{F max}} = \frac{2600 Ω}{500 Ω} = 5.2$$

Applying the correlation table with the degree of steatosis on the factor Z—real obtained, the inventive apparatus for measuring steatosis obtained the following indicative value of the degree of steatosis: 25%-60% (MODERATE STEATOSIS).

### Use in Assistance in Discarding Organs

Applying the results of the two cases described in the example of FIG. 7 to the situation of liver transplant, with the results obtained it will be determined that the liver of the second case is affected with moderate steatosis and would not be a good candidate for transplant donation. This use of the inventive apparatus for measuring steatosis can be fully extrapolated to the case of liver transplant in humans, since it is only necessary to obtain the required interpolation tables by means of a clinical study of transplant organs. Having an indicator of the degree of steatosis in a way that is objective and quick therefore permits an informed decision to be taken, which would be impossible to take if a biopsy had to be performed and the histological results waited for, with a considerable improvement in reliability and speed in a field of critical importance in health and in which time plays a crucial role.

What is claimed:

1.-20. (canceled)

21. An apparatus for clinical used in the diagnosis or monitoring of hepatic steatosis, the apparatus being configured to measure the electrical impedance of a liver and to determine a degree of steatosis as a function of the measured electrical impedance.

22. The apparatus according to claim 21, comprising:
a) an impedance measurement sensor having biocompatible electrodes;
b) a measurement module configured to measure an electrical impedance at a generated frequency between 10 Hz and 10 MHz; and
c) an electronic device configured to control to apparatus and analyze the measurements.

23. The apparatus according to claim 22, further comprising means for measuring hepatic electrical impedance at a plurality of frequencies.

24. The apparatus according to claim 22, further comprising electrodes as non-invasive surface sensors.

25. The apparatus according to claim 22, further comprising electrodes places in small needles as minimally invasive penetrating sensors.

26. The apparatus according to claim 22, wherein the impedance measurement sensor has one on more electrodes comprising gold, silver, silver chloride, platinum or another biocompatible material having high conductance.

27. The apparatus according to claim 24, wherein the impedance measurement sensor has between 2 and 4 electrodes.

28. The apparatus according to claim 25, wherein the impedance measurement sensor has between 2 and 4 electrodes.

29. The apparatus according to claim 26, wherein the impedance measurement sensor has between 2 and 4 electrodes.

30. The apparatus according to claim 24, wherein the impedance measurement sensor has more than 4 electrodes provided on a semi-rigid or flexible support, the electrodes being automatically selectable in groups of two, three or four for carrying out impedance measurements between pairs of the selected electrodes.

31. The apparatus according to claim 25, wherein the impedance measurement sensor has more than 4 electrodes provided on a semi-rigid or flexible support, the electrodes being automatically selectable in groups of two, three or four for carrying out impedance measurements between pairs of the selected electrodes.

32. The apparatus according to claim 26, wherein the impedance measurement sensor has more than 4 electrodes provided on a semi-rigid or flexible support, the electrodes being automatically selectable in groups of two, three or four for carrying out impedance measurements between pairs of the selected electrodes.

33. The apparatus according to claim 24, further comprising a single devices for manually handling the electrodes during a laparotomy.

34. The apparatus according to claim 25, further comprising a single devices for manually handling the electrodes during a laparotomy.

35. The apparatus according to claim 26, further comprising a single devices for manually handling the electrodes during a laparotomy.

36. The apparatus according to claim 22, wherein the electrodes incorporate medical devices of laparoscopy, and are configured for being located in probes or catheters that incorporate the laparoscopy devices for introduction into abdominal cavities during laparoscopy operations.

37. The apparatus according to claim 22, wherein the electrodes are provided on a platform that is configured to weigh the liver when it has been extracted from a body during a transplant procedure associated with forensic medicine or other procedures associated with explanted organs.

38. The apparatus according to claim 21, further comprising:
temperature sensors; and
a module for measuring temperature, wherein
temperature measurements are used to adjust the impedance measurements such that they are independent of temperature.

39. The apparatus according to claim 24, further comprising:
a pressure sensor; and
a module for measuring pressure; wherein
pressure measurements are used to adjust the impedance measurements such that they are independent of pressure between the pressure sensor and the liver.

40. A method of using the apparatus of claim 2, the method comprising the step of diagnosing or monitoring a degree of steatosis in the liver.

41. The method of claim 40, wherein the liver is a human liver.

42. The method of claim 40, wherein the liver is an animal liver.

43. The method of claim 40, wherein the diagnosis or monitoring step further comprises the step of detecting and determining a degree of hepatic steatosis during a surgical transplant operation, wherein the detecting and determining step is performed on a liver that is in vivo or ex vivo.

44. The method of claim 40, wherein the diagnosis or monitoring step further comprises the step of detecting and determining a degree of hepatic steatosis during a laparotomy.

45. The method of claim 40, wherein the diagnosis or monitoring step further comprises the step of detecting and determining a degree of hepatic steatosis during a procedure of forensic medicine.

46. The method of claim 40, wherein the diagnosis or monitoring step further comprises the step of detecting and determining a degree of hepatic steatosis during a food control procedure.
